(19) United States
(12) Patent Application Publication
(10) Pub. No.: US 2007/0250004 A1
(43) Pub. Date: Oct. 25, 2007

(54) SAFE SYRINGE WITH AUTOMATICALLY RETRACTABLE NEEDLE

(76) Inventor: Chung-Hao Tung, Taipei (TW)

Correspondence Address: LOWE HAUPTMAN HAM & BERNER, LLP, 1700 DIAGONAL ROAD, SUITE 300, ALEXANDRIA, VA, 22314, US

(21) Appl. No.: 11/736,864

(22) Filed: Apr. 18, 2007

Related U.S. Application Data
(63) Continuation-in-part of application No. 10/885,792, filed on Jul. 8, 2004.

(51) Int. Cl. A61M 5/32

(52) U.S. Cl. 604/110

(57) ABSTRACT

A safe syringe with automatically retractable needle comprises a cylinder with a holding portion, a needle seat which is fixed within the front end of the cylinder via the holding portion and has a needle holder centrally and a plurality of ribs laterally, and a pusher plunger having a chamber with negative pressure and a constricted opening on the front end thereof. On the edge of the constricted opening is formed a cutting part. And a piston is provided to cover the opening of the pusher plunger. When the pusher plunger is pushed toward the front end of the cylinder, the piston will be cut out and the holding portion will be compressed and deformed to release the needle seat so that the chamber with negative pressure communicates with outside and the detached needle seat can be drawn into the plunger together with a needle by the pressure difference.

### CROSS-REFERENCE TO RELATED DOCUMENTS

The present invention is a continuation in part (CIP) to a U.S. patent application Ser. No. 10/885,792 entitled “Safe syringe with automatic retractable needle” filed on Jul. 8, 2004.

### BACKGROUND OF THE INVENTION

1. Field of the invention

The present invention is related to a safe syringe with an automatically retractable needle and, more especially, to a safe syringe which is provided with a chamber with a negative pressure for withdrawing the needle into the pusher plunger, structurally simple, convenient for operation, has no safety problem concerning the needle dropping out of the cylinder, and is suitable in medical use.

2. Description of the Prior Art

During medical treatment, it is a common therapy to inject medicines into the bodies of patients for controlling the situations of diseases, and the injection of medicines with syringes has been the most effective mode of therapy and has been adopted by the medical field. For used needles have contact with patients, if the diseases of the patients are infective, the needles become infective too. It must be very cautious and careful in dealing with such needles to prevent medical staffs or personnel who discard wastes from being hurt and infected by the infective needles.

As shown in FIG. 8, a needle “b” of a syringe “a” is conventionally only covered by a cover “c” to prevent one from being hurt. However, the cover “c” is designed in accordance with the needle “b”, and its opening for slipping over the needle “b” is quite small. Thereby, it shall be accurately aligned with the needle “b” for slipping over the needle “b”. For the safety of medical staffs, syringes with various retractable type needles and protecting means for injection needles have been developed. As shown in FIG. 9, a conventional retractable type syringe “d” allows a medical staff to push a plunger “f” of its cylinder “e” to an uppermost position to draw a needle “g” back into the cylinder “e”, in order that the syringe “d” discarded after use can be handled with safety.

However, safe syringes with retractable type needles in the markets presently still have the following defects although they can achieve the object of drawing back the needles:
1. A safe syringe with a retractable type needle mainly provides a means for mutual engaging (such as an engaging hook with an engaging detent) between a needle seat and a plunger. When a piston of the plunger is pushed to uppermost position, the engaging hook is very easy to engage with the engaging detent. After the two are engaged with each other, the needle seat and the needle can be together withdrawn into a cylinder of the syringe. This structural design and the production are very complicated, and the costs of them are relatively high.
2. When in use, every medical staff must use both hands to draw back the needle seat and the needle into the cylinder, and the plunger can be pulled out together with the needle directly by an unduly exerted force. Such syringes are inconvenient and dangerous in use. Moreover, when the syringes are discarded randomly or any staff deals with the discarded syringes in a careless way to render the plunger to be pushed out of the cylinder after the needle has been withdrawn into the cylinder, it is meaningless to use the retractable type needle.
3. After the safe syringe with the retractable type needle is used, most part of the plunger will be exposed outside for it is pulled out of the cylinder. The total length of the syringe is abruptly increased, and the total volume of the syringe to be handled is also increased.

In view of these, the inventor develops a safe syringe with an automatically retractable needle to overcome the defects resided now popularly in the above stated safe syringes with retractable type needles.

### SUMMARY OF THE INVENTION

A primary object of the present invention is to provide a safe syringe with an automatically retractable needle, wherein a pressure difference is generated to withdraw a needle automatically into a pusher plunger.

Another object of the present invention is to provide a safe syringe with an automatically retractable needle that is structurally simple, convenient for operation, and there has no worry concerning safety problem of needle dropping out of the cylinder after drawing back.

In order to achieve the above objects, the present invention provides a safe syringe with an automatically retractable needle, and the syringe comprises a cylinder, a needle seat, a pusher plunger, a piston, and a cutting part. The cylinder is a hollow barrel. The needle seat is connected to the front end of the cylinder for holding a needle. The pusher plunger is a hollow rod adapted for slipping in the cylinder, and able to move forwards and rearwards in the cylinder to draw in medicine for injection, wherein the interior of the pusher plunger is a chamber with a negative pressure, and the front end of the pusher plunger is disposed with the piston. The cutting part is used for cutting the piston in the front end of the pusher plunger when the pusher plunger is pushed forwards. Thereby, the chamber with negative pressure communicates with outside, and the needle seat together with a needle are withdrawn into the pusher plunger by the pressure difference.

The present invention will be apparent after reading the detail description of the preferred embodiments thereof in reference to the accompanying drawings.

### BRIEF DESCRIPTION OF THE DRAWINGS

FIG. 1 is an exploded perspective view showing the safe syringe with automatically retractable needle of an embodiment of the present invention

FIG. 2 is a schematic sectional view of the embodiment of the present invention after being assembled;

FIG. 3 is a schematic sectional view of the embodiment of the present invention showing a cylinder contains medicine before injection;

FIG. 4 is a schematic sectional view of the embodiment of the present invention showing the state that an annular sharp edge pierces a piston;

FIG. 5 is a schematic sectional view of the embodiment of the present invention showing that a needle seat is released from a holding portion;

FIG. 6 is a schematic sectional view of the embodiment of the present invention showing the needle seat is withdrawn into a pusher plunger together with a needle;

FIG. 7 is a perspective view of the embodiment of the present invention showing the pusher plunger;

FIG. 8 is a sectional view showing a conventional needle slipped over with a cover; and

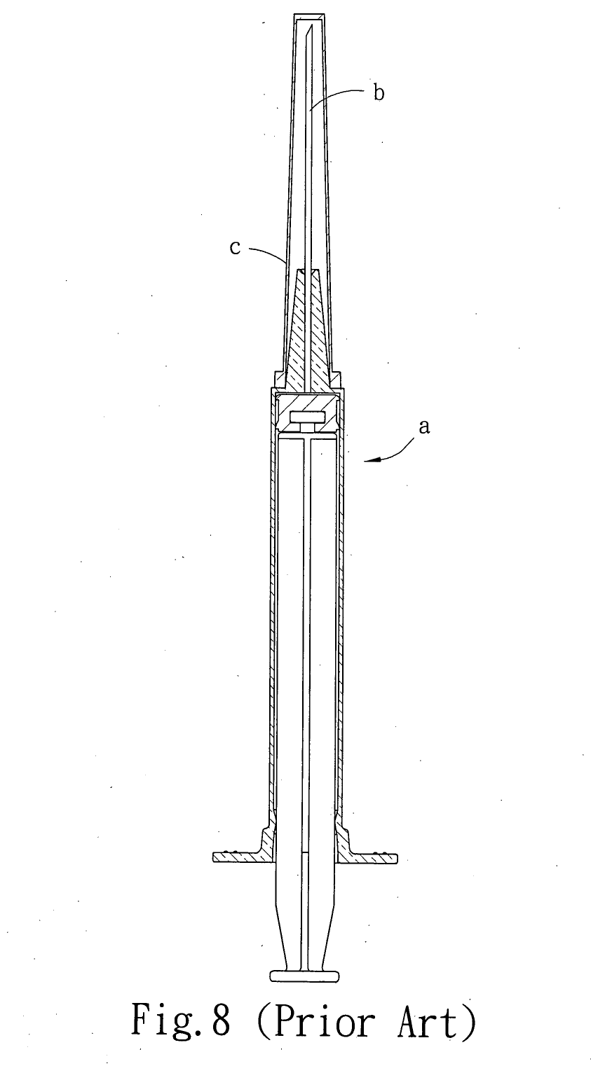

FIG. 9 is a schematic sectional view of a conventional retractable type safe syringe showing the needle withdrawn back into a cylinder in use.

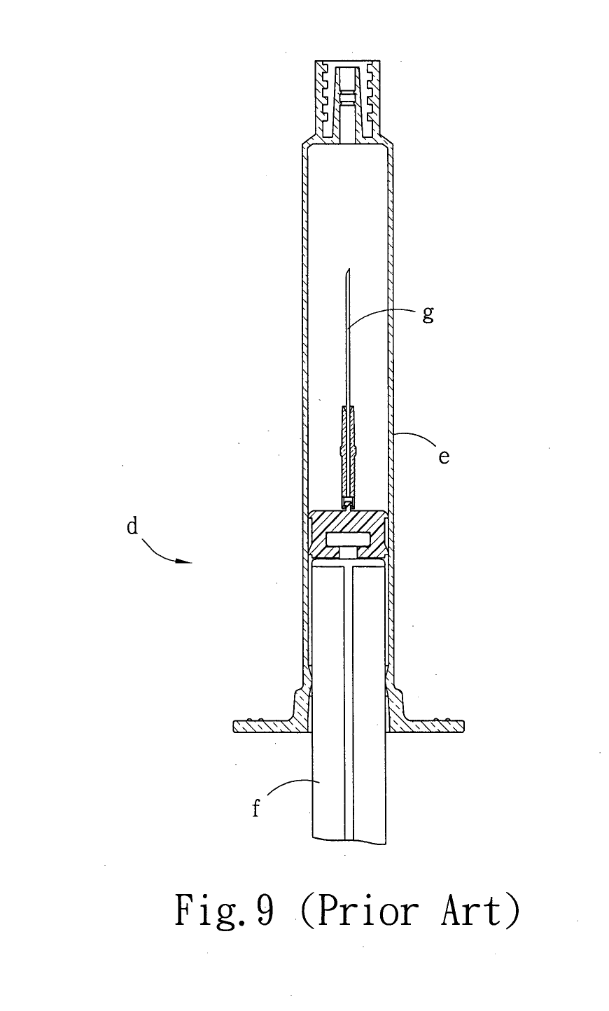

### DETAILED DESCRIPTION OF THE PREFERRED EMBODIMENT

The present invention provides a safe syringe with an automatically retractable needle, and the syringe comprises a cylinder 1, a needle seat 2, a pusher plunger 3, a piston 34, and a cutting part 331. The cylinder 1 is a hollow barrel. The needle seat 2 is connected to the front end of the cylinder 1 for holding a needle 24. The pusher plunger 3 is a hollow rod adapted for slipping in the cylinder 1, and able to move forwards and rearwards in the cylinder 1 to draw in medicine for injection, wherein the interior of the pusher plunger 3 is a chamber 31 with a negative pressure, and the front end of the pusher plunger 3 is disposed with the piston 34. The cutting part 331 is used for cutting the piston 34 in the front of the pusher plunger 3 when the pusher plunger 3 is pushed forwards. Thereby, the chamber 31 of negative pressure communicates with outside, and the needle seat 2 together with the needle 24 are withdrawn into the pusher plunger 3 by the pressure difference.

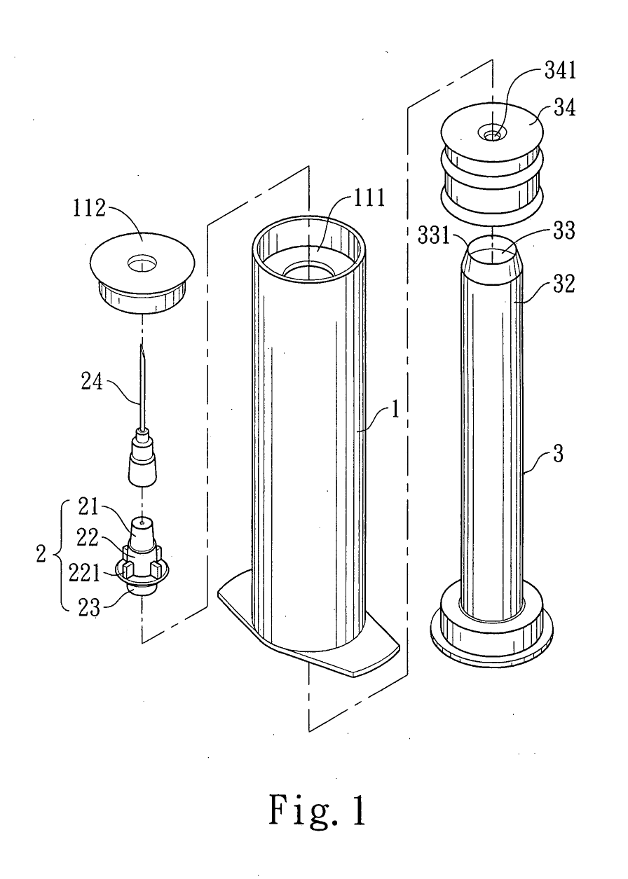

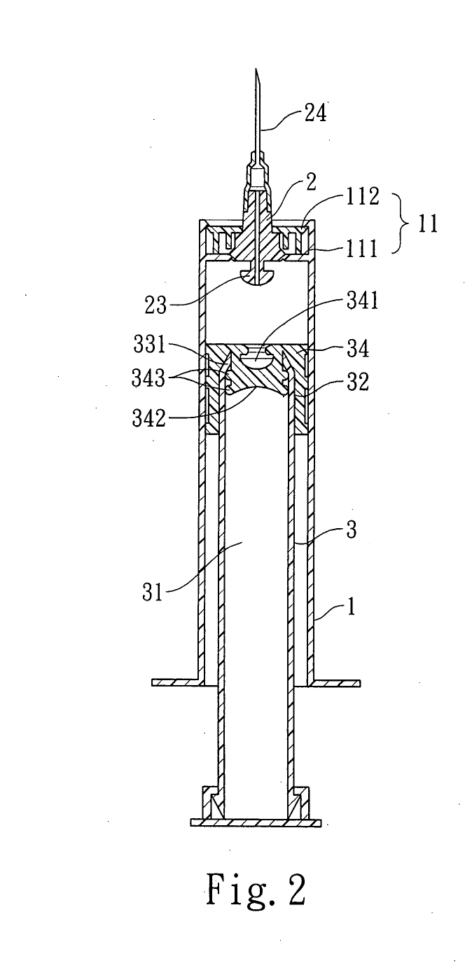

The figures of FIG. 1-7 show an embodiment according to the present invention. As shown in FIG. 1-2, the safe syringe with an automatically retractable needle comprises a cylinder 1, a needle seat 2, and a pusher plunger 3.

The cylinder 1 is a hollow barrel and has a holding portion 11 within the front end thereof. The holding portion 11 includes a base 111 and a holding member 112, wherein the base 111 is used for supporting the needle seat 2 and the holding member 112 is used for holding the needle seat 2.

As mentioned above, the needle seat 2 is firmly held in the front end of the cylinder 1 via the holding portion 11 and includes a needle holder 21, a body 22, and a bottom block 23. The needle holder 21 is on the central upper portion of the needle seat 2 for holding the external needle 24. Besides, the body 22 of the needle seat 2 has a plurality of ribs 221, which is equidistantly arranged on the periphery of the body 22.

The pusher plunger 3 is a hollow rod adapted for slipping in the cylinder 1 and able to move forwards and rearwards in the cylinder 1 to draw in medicine for injection. The interior of the pusher plunger 3 is a chamber 31 with a negative pressure. Besides, the pusher plunger 3 has a constricted opening 33 in its front end and on the edge of the opening 33 forms the cutting part 331, which is an annular sharp edge. The piston 34 is provided to envelope the constricted opening 33 and part of the piston 34 extends into the interior of the pusher plunger 3. The surface of the part of the piston 34 extending into the interior of the pusher plunger 3 is designed as a curved surface 342 for generating a balanced expansion pressure within the chamber 31 of a negative pressure to seal the chamber 31 securely. Besides, the part of the piston 34 extending into the pusher plunger 3 contacts the inner surface of the pusher plunger 3 via a plurality of protrusions 343.

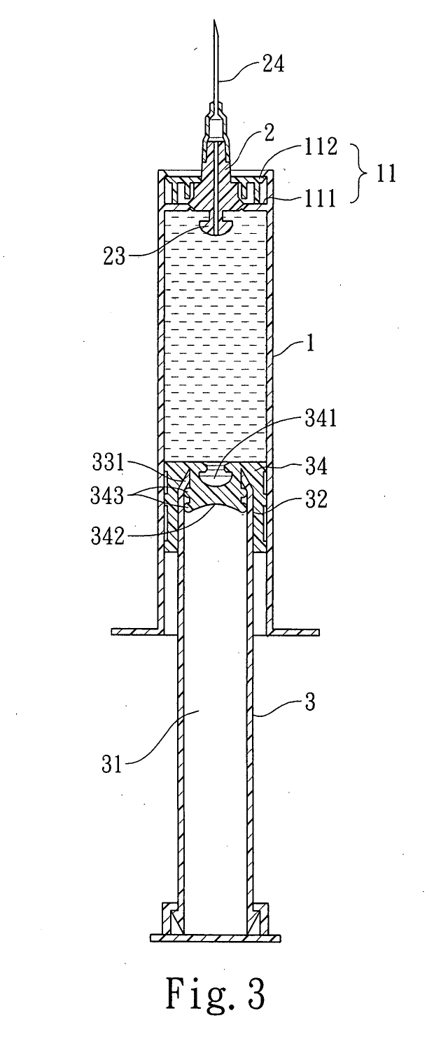

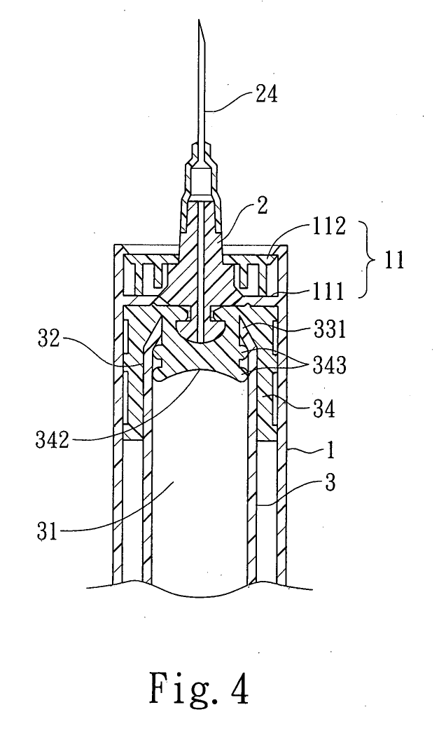

As shown in FIG. 3-4, when in practice, after completing the injection of medicines and drawing the syringe out of a patient's body, the pusher plunger 3 can be further pushed to the front end of the cylinder 1. Then a recess 341 of the piston 34 is connected with the bottom 23 of the needle seat 2 first. If the pusher plunger 3 is pushed forward continuously, the cutting part 331 on the edge of the opening 33 will cut out the piston 34 and then compress and deform the holding portion 11 to release the needle seat 2. By the destruction of the piston 34 and the deformation of the holding portion 11 via the cutting part 331, the chamber 31 with a negative pressure communicates with outside.

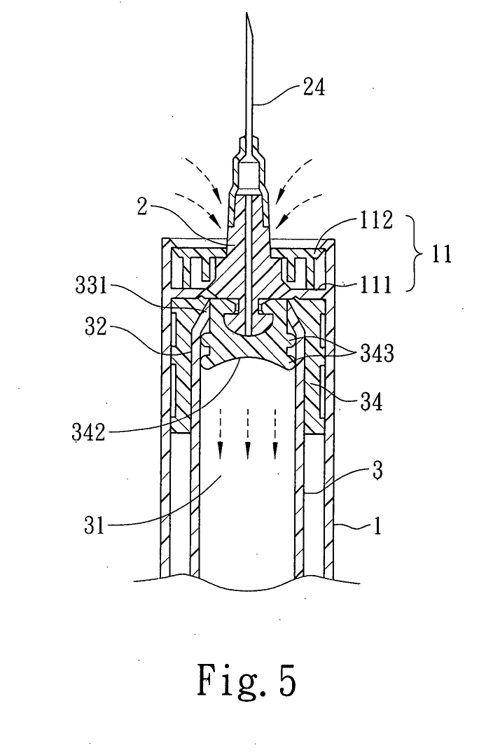

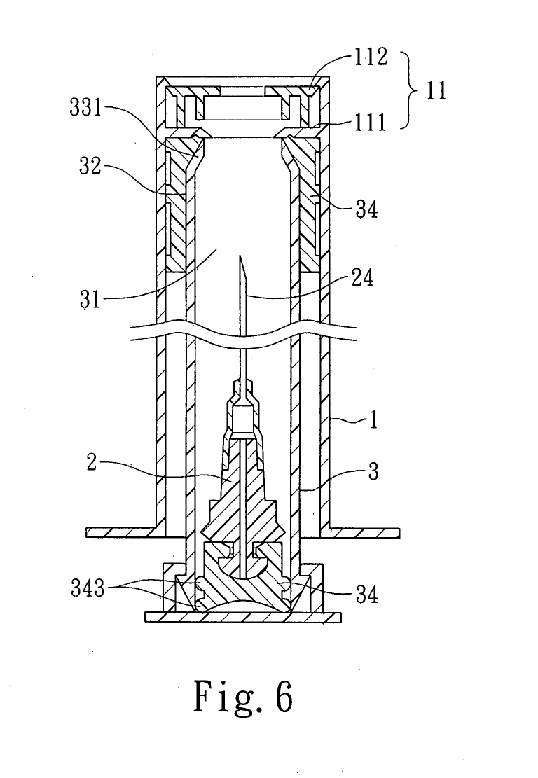

As shown in FIG. 5, because the outside pressure is higher than the pressure inside the chamber 31, the pressure difference will produce a push force from outside into the inside of the chamber 31. Thereby, as shown in FIG. 6, the detached needle seat 2 with the needle 24 is withdrawn into the pusher plunger 3 together with the cut piston by the pressure difference.

Moreover, the detached needle seat 2 can be more smoothly withdrawn into the pusher plunger 3 via the plurality of ribs 221. In addition, the piston 34 contacts with the inner surface of the pusher plunger 3 via a plurality of protrusions 343, by which the cut piston together with the needle seat 2 and the needle 24 can be more smoothly withdrawn into the pusher plunger 3.

Furthermore, by the constricted design of the opening 33, the released needle seat 2 can be surely withdrawn into the pusher plunger 3 rather than into the cylinder 1 but outside the pusher plunger 3. And the combination of the recess 341 of the piston 34 and the bottom 23 of the needle seat 2 can work together with the negative pressure within the chamber 31 of the pusher plunger 3 to withdraw the released needle seat 2 with the needle 24 together with the cut piston into the pusher plunger 3.

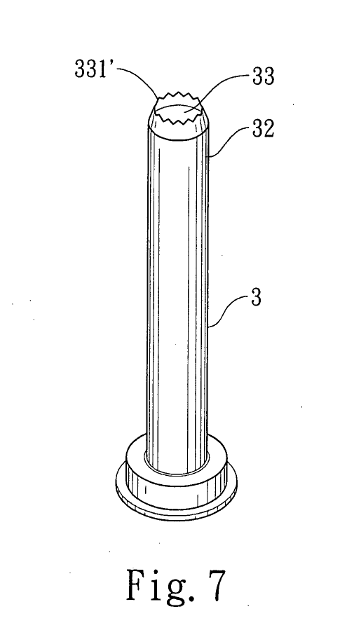

In addition, as shown in FIG. 7, the cutting part 331′ disposed on the edge of the opening 33 of the pusher plunger 3 is a serrated edge used for cutting the piston 34 more effectively.

Thereby, the present invention has the following advantages:
1. The present invention provides a piston enveloping the opening of the pusher plunger, which is formed as a unity to decrease the cost of material in assembly.
2. The present invention provides a cutting part on the edge of a constricted opening for cutting the piston, and by the constricted design of the opening, the released needle seat can be surely withdrawn into the pusher plunger rather than into the cylinder but outside the pusher plunger.
3. The present invention provides a holding portion within the front end thereof, by which the needle seat can be securely connected with the cylinder or released from the holding portion simply by exerting pressure.
4. The present invention provides a piston with a curved surface facing the interior of the pusher plunger, by which a balanced expansion pressure within the chamber with negative pressure can be generated to seal the chamber securely.
5. The present invention provides a piston contacting the inner surface of the pusher plunger via a plurality of protrusions, by which the cut piston together with the needle seat and the needle can be more smoothly withdrawn into the pusher plunger.

Accordingly, as disclosed in the above description and attached drawings, the present invention can provide a safe syringe with an automatically retractable needle. It is new and can be put into industrial use.

While the invention has been described with reference to certain embodiments, it will be understood by those skilled in the art that various changes may be made and equivalents may be substituted without departing from the scope of the present invention.

What is claimed is:

1. A safe syringe with an automatically retractable needle, comprising
a cylinder, which is a hollow barrel;
a needle seat connected to the front end of the cylinder for holding a needle;
a pusher plunger, which is a hollow rod adapted for slipping in the cylinder, and able to move forwards and rearwards in the cylinder to draw in medicine for injection, wherein the interior of the pusher plunger is a chamber with a negative pressure, the front end of the pusher plunger is disposed with a piston; and
a cutting part for cutting the piston in the front of the pusher plunger when the pusher plunger is pushed forwards;
wherein the chamber with negative pressure communicates with outside, and the needle seat together with the needle are withdrawn into the pusher plunger by the pressure difference.

2. The safe syringe with an automatically retractable needle as claimed in claim 1, wherein a holding portion is further provided in the front end of the cylinder for connecting the needle seat, and after cutting the piston, the cutting part compresses and deforms the holding portion to release the needle seat to be withdrawn into the pusher plunger.

3. The safe syringe with an automatically retractable needle as claimed in claim 2, wherein the holding portion further includes a holding member and a base, in which the holding member is used for holding the needle seat and the base is fixed within the front end of the cylinder.

4. The safe syringe with an automatically retractable needle as claimed in claim 3, wherein the needle seat further includes a needle holder, a body, and a bottom block, in which the bottom block is used for connecting the piston, and a plurality of ribs are equidistantly arranged on the periphery of the body for smoothly withdrawing the needle seat into the pusher plunger.

5. The safe syringe with an automatically retractable needle as claimed in claim 3, wherein a constricted opening is provided in the front end of the pusher plunger, and on the edge of the opening forms the cutting part, which is an annular sharp edge used for cutting the piston to communicate the chamber of a negative pressure with outside when the pusher plunger is pushed forward.

6. The safe syringe with an automatically retractable needle as claimed in claim 5, wherein the edge of the opening is a serrate edge.

7. The safe syringe with an automatically retractable needle as claimed in claim 5, wherein part of the piston covers the opening of the pusher plunger, and another part of the piston extends into the opening, by which the pusher plunger is sealed.

8. The safe syringe with an automatically retractable needle as claimed in claim 7, wherein a recess is further provided centrally on the outside surface of the piston for connecting the bottom block of the needle block.

9. The safe syringe with an automatically retractable needle as claimed in claim 8, wherein the part of the piston extending into the pusher plunger laterally has a plurality of protrusions used for smoothly withdrawing the cut piston connected with the needle seat into the pusher plunger.

10. The safe syringe with an automatically retractable needle as claimed in claim 8, wherein the part of the piston extending into the pusher plunger has a curved surface for generating a balanced expansion pressure within the chamber with negative pressure to seal the chamber.

11. The safe syringe with an automatically retractable needle as claimed in claim 1, wherein the needle seat further includes a needle holder, a body, and a bottom block, in which the bottom block is used for connecting the piston, and a plurality of ribs are equidistantly arranged on the periphery of the body for smoothly withdrawing the needle seat into the pusher plunger.

12. The safe syringe with an automatically retractable needle as claimed in claim 1, wherein a constricted opening is provided in the front end of the pusher plunger, and on the edge of the opening forms the cutting part, which is an annular sharp edge used for cutting the piston to communicate the chamber of a negative pressure with outside when the pusher plunger is pushed forward.

13. The safe syringe with an automatically retractable needle as claimed in claim 12, wherein the edge of the opening is a serrate edge.

14. The safe syringe with an automatically retractable needle as claimed in claim 12, wherein part of the piston covers the opening of the pusher plunger, and another part of the piston extends into the opening, by which the pusher plunger is sealed.

15. The safe syringe with an automatically retractable needle as claimed in claim 14, wherein a recess is further provided centrally on the outside surface of the piston for connecting the bottom block of the needle block.

16. The safe syringe with an automatically retractable needle as claimed in claim 15, wherein the part of the piston extending into the pusher plunger laterally has a plurality of protrusions used for smoothly withdrawing the cut piston connected with the needle seat into the pusher plunger.

17. The safe syringe with an automatically retractable needle as claimed in claim 15, wherein the part of the piston extending into the pusher plunger has a curved surface for generating a balanced expansion pressure within the chamber with negative pressure to seal the chamber.
